(19) United States
(12) Patent Application Publication
(10) Pub. No.: US 2023/0190522 A1
(43) Pub. Date: Jun. 22, 2023

(54) DEVICE FOR DIAGNOSING AND TREATING OCULAR DEVIATION

(71) Applicant: AJOU UNIVERSITY INDUSTRY-ACADEMIC COOPERATION FOUNDATION, Suwon-si (KR)

(72) Inventor: Ji Man HONG, Yongin-si (KR)

(73) Assignee: AJOU UNIVERSITY INDUSTRY-ACADEMIC COOPERATION FOUNDATION, Suwon-si (KR)

(21) Appl. No.: 17/926,425

(22) PCT Filed: May 18, 2021
(86) PCT No.: PCT/KR2021/006185

§ 371 Date: Nov. 18, 2022

(30) Foreign Application Priority Data
May 19, 2020 (KR) 10-2020-0059442

(51) Int. Cl. A61F 7/12; A61F 7/00

(52) U.S. Cl. CPC A61F 7/12; A61F 7/0085; A61F 7/007; A61B 3/113

(57) ABSTRACT

The present invention provides a device for diagnosing and treating ocular deviation, comprising: an eye mask which is worn by an examinee, and in which an eye movement capturing means for capturing the eye movement of the examinee is provided; a temperature-regulating pipe which is inserted into the ear of the examinee and which increases or decreases the temperature inside the ear of the examinee; a heat source supply part for supplying a heat source to the temperature-regulating pipe from the outside in order to increase and decrease the temperature of the temperature-regulating pipe; a fixing main body which is worn by the examinee, and which has a mounting hole, in which the temperature-regulating pipe is mounted, in order to fix the temperature-regulating pipe inserted into the ear of the examinee; and a control part, which is connected to the eye movement capturing means and the heat source supply part, receives an image of the eye captured by the eye movement capturing means, and extracts center coordinates from the transmitted image of the eye to compare same with the center coordinates of the eye inputted in advance, and then operates the heat source supply part to control the temperature of the temperature-regulating pipe in order to move the eye to the center coordinates of the eye inputted in advance.

### TECHNICAL FIELD

The present invention relates to a device for diagnosing and treating ocular deviation, and more particularly, to a device for diagnosing and treating ocular deviation, capable of treating the symptoms of patients with dizziness and stroke patients by measuring the degree of ocular deviation in patients with vestibular abnormalities or dizziness and stroke patients and moving the deviated eye to one side through temperature stimulation.

### BACKGROUND ART

Human eyes are based on conjugate gaze which focuses on an image by moving different muscles in order to see a desired object or see an object accurately. In addition, the horizontal gaze center is also controlled in a frontal eye field at a higher level than this. In convulsions in which the excitability of the frontal eye field is increased, the eye is horizontally deviated in an opposite direction to the convulsing hemisphere. In contrast, in strokes in which the function is impaired due to decreased blood flow, the eye is horizontally deflected in the direction of the stroke hemisphere. This is also used to localize clinical neurological lesions based on the phenomenon of conjugate gaze. Neurologically, for the horizontal ocular deviation, the central center connecting the eye muscles with different directions is required. The horizontal conjugate gaze is controlled by the paramedian pontine reticular formation (PPRF) of the ipsilateral brain stem. Finally, due to the stimulation of the PPRF region, the horizontal conjugate gaze deviation is caused in the eye toward the stimulated side.

Caloric testing is an important test to check unilateral vestibular function and is mainly used to evaluate patients with dizziness. That is, in the caloric testing, after putting cold or hot water different from the human body temperature (about 37° C.) into the ear, the convection of the endolymph fluid is made different on both sides. Then, the vestibular function of the unilateral side is checked based on the physiological mechanism that the stimulation of different PPRFs on both sides (right and left sides) by temperature stimulation causes the eye to be deviated and nystagmus occurs in the opposite direction of the deviation. The horizontal conjugate muscle movement of the eye induced by the temperature stimulation of the inner ear using cold or hot water can be used as a medical reflex reaction to know the function of the brain stem. Therefore, in clinically examining the function of the middle brain stem, the caloric testing is an essential reflex reaction and is known as the most basic test to check the brain stem reflex reaction of brain-dead patients along with the pupil reflex reaction and the corneal reflex reaction.

In general, eye movements have been recorded with various stimuli by putting on an eye movement test mask in a state of being in the dark or out of the focus with a magnifying glass, and there was also an a method of performing testing and analysis by putting 20° C. water in a 20 ml syringe and delivering the temperature for 20 seconds. In addition, a method of perfusion for 40 seconds in one ear using 30° C. and 44° C. water, which were different from each other and were 7° C. lower or higher than body temperature, was also presented.

However, since the caloric testing is generally performed using water, its use is inevitably limited for patients with no eardrum or impaired ear function. It is true that the patient is very reluctant to undergo the examination because cold or hot water, which has a very strong foreign body sensation, is put into the ear for the examination. Due to these limitations, the caloric testing has been developed in the form of stimulation and interpretation using open air. However, there occurs a problem in that, since air is supplied into the ear, the patient may feel a sense of rejection due to the feeling of a foreign body, and the temperature inside the ear of the examinee may not be precisely controlled.

The technology that is the background of the present invention is disclosed in Korean Patent Registration No. 10-0336167 (registered on Apr. 29, 2002).

### DISCLOSURE

### Technical Problem

The present invention has been made in an effort to solve the above problems, and an object of the present invention is to provide a device for diagnosing and treating ocular deviation, wherein, after a temperature-regulating pipe having a double pipe structure with high thermal conductivity is inserted without injecting water or air into an ear of an examinee, a control part adjusts the temperature of the temperature-regulating pipe according to the condition of the examinee by using a heat source supply part, thereby reducing the reluctance of the examinee, and the temperature inside an ear of a patient with horizontal ocular deviation caused by permanent cerebral dysfunction is precisely regulated by using the control part and the temperature-regulating pipe, so that the horizontally deviated eye is moved by stimulating the paramedian pontine reticular formation (PPRF) of the brain stem with cold or hot temperatures while measuring the degree of ocular deviation of the patient.

### Technical Solution

In order to achieve the above objects, the present invention provides a device for diagnosing and treating ocular deviation, including: an eye mask which is worn by an examinee, and in which an eye movement capturing means for capturing the eye movement of the examinee is provided; a temperature-regulating pipe which is inserted into the ear of the examinee and which increases or decreases the temperature inside the ear of the examinee; a heat source supply part for supplying a heat source to the temperature-regulating pipe from the outside in order to increase and decrease the temperature of the temperature-regulating pipe; a fixing main body which is worn by the examinee, and which has a mounting hole, in which the temperature-regulating pipe is mounted, in order to fix the temperature-regulating pipe inserted into the ear of the examinee; and a control part, which is connected to the eye movement capturing means and the heat source supply part, receives an image of the eye captured by the eye movement capturing means, and extracts center coordinates from the transmitted image of the eye to compare same with the center coordinates of the eye inputted in advance, and then operates the heat source supply part to control the temperature of the temperature-regulating pipe in order to move the eye to the center coordinates of the eye inputted in advance.

In the device for diagnosing and treating ocular deviation according to the present invention, the eye mask may include: a mask main body which is worn around eyes of the examinee and which provides a dark environment for capturing eye movement; an eye movement capturing means which is provided inside the mask main body and which captures the eye movement; and a fixing means which is connected to the mask main body and which fixes the mask main body to a head of the examinee. The eye movement capturing means may use an infrared camera capable of capturing the eye movement even in the dark environment.

The temperature-regulating pipe may include: a first temperature-regulating pipe inserted into one ear of the examinee; and a second temperature-regulating pipe inserted into the other ear of the examinee. The first temperature-regulating pipe and the second temperature-regulating pipe may include: an insertion main body which is inserted into the ear of the examinee and which regulates the temperature in the ear of the examinee by transmitting the temperature of the heat source supplied from the heat source supply part; a main body case which surrounds the insertion main body from the outside; and a connecting member which connects the insertion main body and the heat source supply part. The insertion main body and the connecting member may have a double pipe structure.

The insertion main body may include: a main body inlet pipe into which the heat source supplied from the heat source supply part and the connecting member is introduced; and a main body circulation pipe which circulates the heat source introduced into the main body inlet pipe. The connecting member may include: a connection inlet pipe which is connected to the main body inlet pipe and which supplies the heat source supplied from the heat source supply part to the main body inlet pipe; and a connection circulation pipe which is connected to the main body circulation pipe and which circulates the heat source moving through the main body circulation pipe to the heat source supply part. The main body inlet pipe and the connection inlet pipe are located inside the main body circulation pipe and the connection circulation pipe.

The insertion main body may be made of a polymer material having a high heat transfer rate and a coefficient of thermal expansion similar to that of silicone.

The fixing main body may include: a front head plate which is disposed in front of a head of the examinee; a back head plate which is disposed behind the head of the examinee to correspond to the front head plate; a first side connecting plate and a second side connecting plate which are disposed on both sides of the head of the examinee and which connect the front head plate and the rear head plate arranged to be spaced apart from each other; a wearing belt which connects the first side connecting plate and the second side connecting plate; and a fixing frame which is mounted on the first side connecting plate and the second side connecting plate and which fixes the temperature-regulating pipe in a state of being inserted into the ear of the examinee. The fixing frame may be vertically moved from the first side connecting plate and the second side connecting plate.

The fixing frame may include: a mounting body having one end mounted on the first side connecting plate and the second side connecting plate; an elevating rod which is provided to be spaced apart from the other end of the mounting body; an elevating main body which elevates in the elevating rod and has a through hole through which the elevating rod passes; an elevating regulating member which is mounted on both sides of the elevating body and which regulates the elevation of the elevating main body; and a mounting main body which is rotatably mounted to a mounting hole formed in the central portion of the elevating main body and on which the temperature-regulating pipe is mounted.

A through hole may be formed in the central portion of the mounting main body. The mounting main body may include: a mounting end which has a spherical circumferential surface and is rotatably mounted to the mounting hole; a seating end which is connected to one side of the mounting end and on which the temperature-regulating pipe inserted into the through hole is seated; and a separation preventing end which is provided at an end of the seating end and which prevents separation of the temperature-regulating pipe seated on the seating end. The separation preventing end may be rotatably coupled to the seating end. The seating end may be provided with a rotation regulating member for regulating the rotation of the separation preventing end.

### Advantageous Effects

In a device for diagnosing and treating ocular deviation according to the present invention, a control part controls a heat source supply part to regulate a temperature of a temperature-regulating pipe, so that quantitative caloric testing can be performed. Since the temperature-regulating pipe with high thermal conductivity is used instead of water, it is possible to minimize the stimulation to an ear of an examinee. The temperature-regulating pipe inserted into the ear of the examinee can be easily fixed using a fixing main body. Since eye movement is analyzed using an eye movement capturing means and the control part and the temperature inside the ear is controlled using the heat source supply part and the temperature-regulating pipe, repeated rehabilitation treatment is possible by artificially controlling the eye movement of the examinee or patient with ocular deviation. In addition, the symptoms of stroke patients are improved.

### DESCRIPTION OF DRAWINGS

FIG. 1 is a view schematically illustrating a state in which an examinee wears a device for diagnosing and treating ocular deviation according to an embodiment of the present invention.

FIG. 2 is an enlarged view illustrating a configuration of an eye mask illustrated in FIG. 1.

FIG. 3 is a view illustrating a sequence in which the eye mask transmits a captured image to a control part and the control part controls a heat source supply part.

FIGS. 4 and 5 are views illustrating a process in which the control part compares transmitted center coordinates of an eye with center coordinates of the eye inputted in advance and then operates the heat source supply part to move the eye to the right or left side.

FIG. 6 is an enlarged view illustrating a fixing main body illustrated in FIG. 1.

FIG. 7 is an enlarged view illustrating a fixing frame illustrated in FIG. 6.

FIG. 8 is a view illustrating a state in which an elevating body illustrated in FIG. 7 elevates in an elevating rod.

FIG. 9 is a view illustrating a state in which a release preventing end illustrated in FIG. 7 is rotated at a seating end.

FIG. 10 is an enlarged view illustrating a temperature-regulating pipe illustrated in FIG. 1.

### MODES OF THE INVENTION

Hereinafter, preferred embodiments of the present invention will be described in detail with reference to the accompanying drawings. The terms or words used in the present specification and the claims should not be construed as being limited to ordinary or dictionary meanings. The terms or words should be construed as meanings and concepts consistent with the technical idea of the present invention, based on the principle that the inventors can appropriately define the concept of the terms in order to explain their invention in the best way.

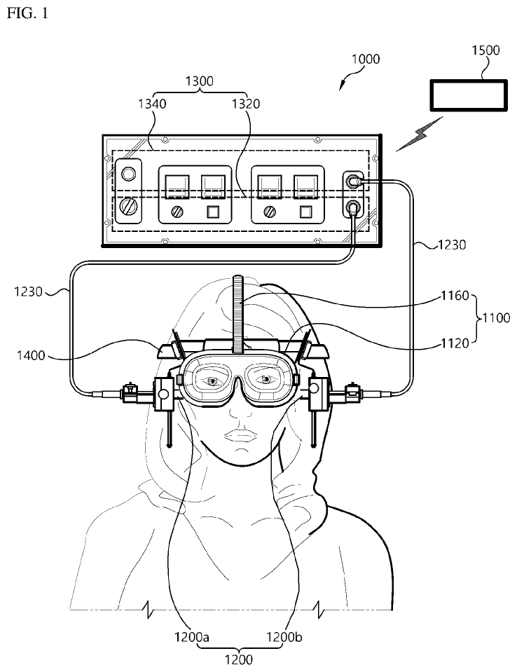

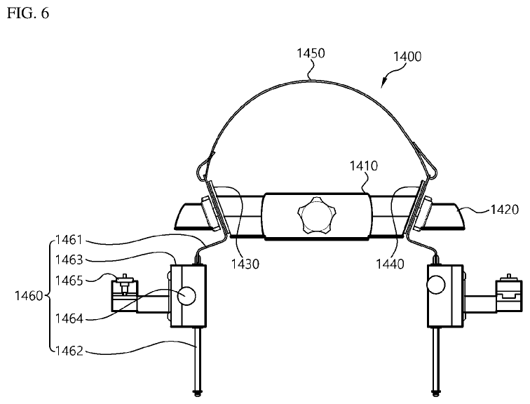

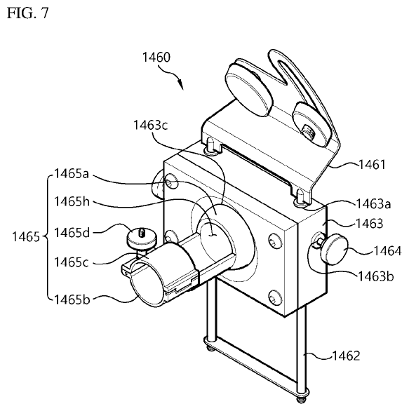

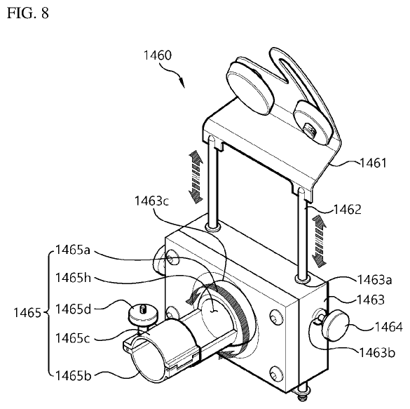

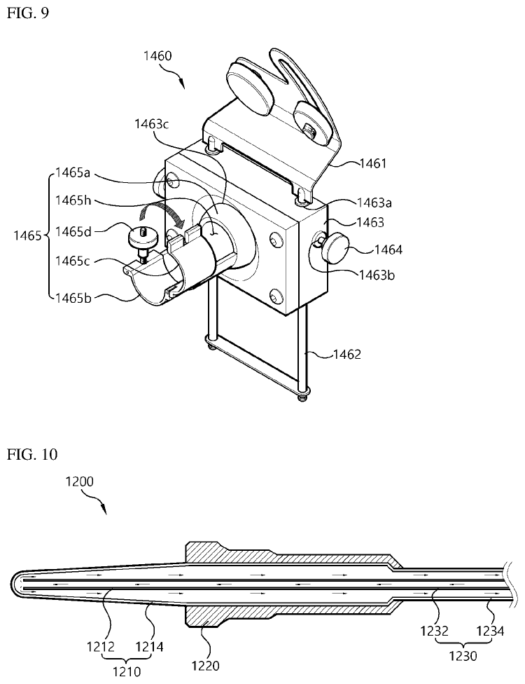

Referring to FIGS. 1 to 10, a device 100 for diagnosing and treating ocular deviation according to an embodiment of the present invention includes an eye mask 1100, a temperature-regulating pipe 1200, a heat source supply part 1300, a fixing main body 1400, and a control part 1500.

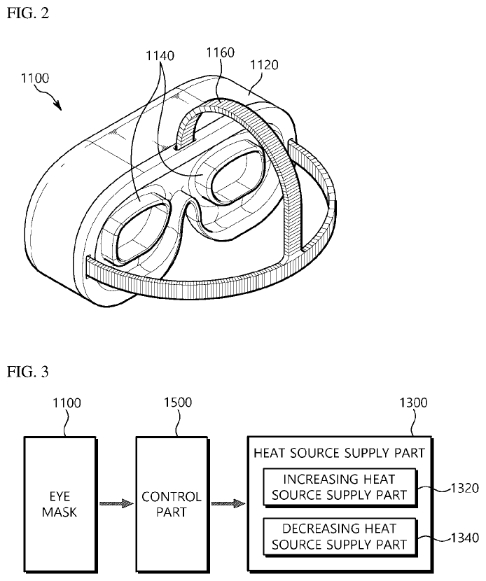

Referring to FIGS. 1 and 2, the eye mask 1100 is worn by an examinee. The eye mask 1100 is provided in a goggle type and serves to measure the ocular deviation of the examinee while blocking the eye field of the examinee from the outside. The eye mask 1100 includes a mask main body 1120, an eye movement capturing means 1140, and a fixing means 1160.

The mask main body 1120 is in close contact with the face of the examinee so that the eye field of the examinee is blocked from the outside. Since the mask main body 1120 is in close contact with the face of the examinee, a dark environment for capturing the eye movement is provided. The eye movement capturing means 1140 is provided inside the mask main body 1120. The eye movement capturing means 1140 captures the eye movement in order to measure the ocular deviation of the examinee in the dark environment. An infrared camera capable of capturing even in the dark environment is preferably used as the eye movement capturing means 1140. The infrared camera is a general camera, and a detailed description thereof will be omitted. Preferably, the eye movement and ocular deviation captured by the eye movement capturing means 1140 are transmitted to the control part 1500 to be described below. The fixing means 1160 is connected to the mask main body 1120 in order to fix the mask main body 1120 to the head of the examinee. A string member is preferably used as the fixing means 1160. However, the present invention is not limited thereto. Various types of fixing means for fixing the mask main body 1120 to the head of the examinee are possible. Here, the string member 116 is a general member, and a detailed description thereof will be omitted.

Referring to FIGS. 1 and 10, the temperature-regulating pipe 1200 is inserted into the ear of the examinee. The temperature-regulating pipe 1200 is inserted into the ear of the examinee to increase or decrease the temperature inside the ear of the examinee.

The temperature-regulating pipe 1200 includes a first temperature-regulating pipe 1200a and a second temperature-regulating pipe 1200b. The first temperature-regulating pipe 1200a and the second temperature-regulating pipe 1200b include an insertion main body 1210, a main body case 1220, and a connecting member 1230.

The first temperature-regulating pipe 1200a is inserted into one ear of the examinee, and the second temperature-regulating pipe 1200b is inserted into the other ear of the examinee to correspond to the first temperature-regulating pipe 1200a. Opposite heat sources supplied from the heat source supply part 1300 to be described below are supplied to the first temperature-regulating pipe 1200a and the second temperature-regulating pipe 1200b.

The insertion main body 1210 is inserted into the ear of the examinee and serves to regulate the temperature inside the ear of the examinee by transferring the temperature of the heat source, which is supplied from the heat source supply part 1300 to be described below, to the ear of the examinee. The insertion main body 1210 is connected to the heat source supply part 1300 to be described below by the connecting member 1230. The insertion main body 1210 and the connecting member 1230 have a double pipe structure. The insertion main body 1210 is surrounded by the main body case 1220.

The insertion main body 1210 includes a main body inlet pipe 1212 and a main body circulation pipe 1214, and the connecting member 1230 includes a connection inlet pipe 1232 and a connection circulation pipe 1234. The heat source supplied from the heat source supply part 1300 to be described below is introduced into the main body inlet pipe 1212 through the connecting member 1230, and the heat source introduced into the main body inlet pipe 1212 is circulated through the main body circulation pipe 1214 and the connecting member 1230 to the heat source supply part 1300 to be described below.

The connection inlet pipe 1232 is connected to the main body inlet pipe 1212 and supplies, to the main body inlet pipe 1212, the heat source supplied from the heat source supply part 1300 to be described below. The connection circulation pipe 1234 is connected to the main body circulation pipe 1214 and supplies, to the heat source supply part 1300 to be described below, the heat source moved through the main body circulation pipe 1214.

The main body inlet pipe 1212 is provided inside the main body circulation pipe 1214, and the connection inlet pipe 1232 is provided inside the connection circulation pipe 1234. A screw thread (not illustrated) is formed in a connecting portion of the main body inlet pipe 1212 and the main body circulation pipe 1214, and a screw groove (not illustrated) is formed in a connecting portion of the connection inlet pipe 1232 and the connection circulation pipe 1234. As the insertion main body 1210 rotates forward or backward, the connection to the connecting member 1230 or the connection release from the connecting member 1230 is performed.

The insertion main body 1210 has high thermal conductivity and is made of a polymer material having the same coefficient of thermal expansion as that of silicone. The insertion main body 1210 is preferably provided with a temperature sensor (not illustrated) for measuring the temperature inside the ear of the examinee, into which the insertion main body 1210 is inserted, and the temperature of the insertion main body 1210. The temperature inside the ear of the examinee and the temperature of the insertion main body 1210, which are measured by the temperature sensor (not illustrated), are transmitted to the control part 150 to be described below.

Since the insertion main body 1210 and the connecting member 1230 have a double pipe structure, the heat source whose temperature is controlled by the control part 150 to be described below is quickly moved from the heat source supply part 1300 to be described below to the insertion main body 1210 and is then drained back to the heat source supply part 1300. Accordingly, the temperature of the heat source in the heat source supply part 1300 is almost the same as the temperature of the heat source measured by the temperature sensor (not illustrated).

The insertion main body 1210 is connected to the heat source supply part 1300 by the connecting member 1230, and the heat source supply part 1300 serves to supply the heat source to the temperature-regulating pipe 1200 in order to increase or decrease the temperature inside the ear of the examinee.

Referring to FIG. 1, the heat source supply part 1300 preferably includes an increasing heat source supply part 1320 and a decreasing heat source supply part 1340. The increasing heat source supply part 1320 serves to supply a hot heat source to the connecting member 1230 in order to increase the temperature of the insertion main body 1210, and an electric heater or the like is preferably used as the supplied hot heat source. The present invention is not limited to the electric heater, and hot water or steam may be used.

The decreasing heat source supply part 1340 serves to supply a cold heat source to the connecting member 1230 in order to decrease the temperature of the insertion main body 1210, and a refrigerator and a refrigerant are preferably used as the supplied cold heat source. The present invention is not limited thereto, and ice may be used.

The first temperature-regulating pipe 1200a of the temperature-regulating pipe 1200 is connected to the increasing heat source supply part 1320 and is inserted into one ear of the examinee to increase the temperature inside the ear of the examinee by the operation of the control part 1500 to be described below, and the second temperature-regulating pipe 1200b is connected to the decreasing heat source supply part 1340 and is inserted into the other ear of the examinee to increase the temperature inside the ear of the examinee by the operation of the control part 1500 to be described below.

Referring to FIGS. 1 and 6 to 9, the temperature-regulating pipe 1200 is mounted on the fixing main body 1400 worn by the examinee and is thus fixed in a state of being inserted into the ear of the examinee. The fixing main body 1400 includes a front head plate 1410, a back head plate 1420, a first side connecting plate 1430, a second side connecting plate 1440, a wearing belt 1450, and a fixing frame 1460.

The front head plate 1410 is disposed in front of the head of the examinee and is in close contact with the forehead, which is the front of the head of the examinee. The back head plate 1420 is disposed behind the head of the examinee and is in close contact with the back of the head of the examinee. The front head plate 1410 and the back head plate 1420 are connected to each other by the first side connecting plate 1430 and the second side connecting plate 1440 disposed spaced apart from each other on both sides of the head of the examinee.

Since the front head plate 1410 and the back head plate 1420 are connected to each other by the first side connecting plate 1430 and the second side connecting plate 1440, the front head plate 1410, the first side connecting plate 1430, the back head plate 1420, and the second side connecting plate 1440 surround the head of the examinee in a ring shape. The first side connecting plate 1430 and the second side connecting plate 1440 spaced apart from each other are connected to each other by the wearing belt 1450. The wearing belt 1450 is in close contact with the upper portion of the head of the examinee. The wearing belt 1450 prevents the front head plate 1410, the first side connecting plate 1430, the back head plate 1420, and the second side connecting plate 1440 surrounding the head of the examinee from going down to the lower portion of the head of the examinee.

The fixing frame 1460 is mounted on the first side connecting plate 1430 and the second side connecting plate 1440. Due to the fixing frame 1460, the temperature-regulating pipe 1200 is fixed in a state of being inserted into the ear of the examinee. The fixing frame 1460 is vertically moved between the first side connecting plate 1430 and the second side connecting plate 1440.

The fixing frame 1460 includes a mounting body 1461, an elevating rod 1462, an elevating main body 1463, an elevating regulating member 1464, and a mounting main body 1465. One end of the mounting body 1461 is mounted on the first side connecting plate 1430 and the second side connecting plate 1440, and the elevating rod 1462 are provided to be spaced apart at the other end of the mounting body 1461.

The elevating main body 1463 is mounted on the elevating rod 1462 and elevates in the elevating rod 1462. A through hole 1463a through which the elevating rod 1462 passes is formed in the elevating main body 1463 to be spaced apart from the elevating main body 1463.

The elevating regulating member 1464 is mounted on both sides of the elevating main body 1463. The elevating regulating member 1464 serves to regulate the elevation of the elevating main body 1463 that elevates in the elevating rod 1462. The insertion hole 1463b is preferably formed in the elevating main body 1463 in order to mount the elevating regulating member 1464.

A mounting hole 1463c is formed in the central portion of the elevating main body 1463, and the mounting main body 1465 is rotatably mounted to the mounting hole 1463c. A through hole 1465h to which the temperature-regulating pipe 1200 is mounted is preferably formed in the mounting main body 1465.

The mounting main body 1465 includes a mounting end 1465a, a seating end 1465b, and a separation preventing end 1465c. The seating end 1465b is provided with a rotation regulating member 1465d. The mounting end 1465a is rotatably mounted to the mounting hole 1463c. The mounting end 1465a preferably has a spherical circumferential surface so as to be rotatable in the mounting hole 1463c.

The seating end 1465b is connected to one side of the mounting end 1465a. The temperature-regulating pipe 1200 inserted into the through hole 1465h is seated on the seating end 1465b. The seating end 1465b has a semi-cylindrical shape with a hollow inside. The separation preventing end 1465c is provided at the end of the seating end 1465b.

The separation preventing end 1465c has a shape corresponding to the seating end 1465b. The separation preventing end 1465c serves to prevent the temperature-regulating pipe 1200 seated on the seating end 1465b from being separated from the seating end 1465b and to fix the temperature-regulating pipe 1200 to the mounting main body 1465.

The separation preventing end 1465c is rotatably hinged to the seating end 1465b, and the seating end 1465b is provided with the rotation regulating member 1465d for regulating and fixing the rotation of the separation preventing end 1465c.

Referring to FIG. 1, the eye movement capturing means 1140, the temperature sensor (not illustrated) provided in the insertion main body 1210 of the temperature-regulating pipe 1200, and the heat source supply part 1300 are connected to the control part 1500. The image of the eye captured by the eye movement capturing means 1140 is transmitted to the control part 1500. The control part 1500 extracts center coordinates from the transmitted image of the eye and compares the extracted center coordinates with center coordinates inputted in advance.

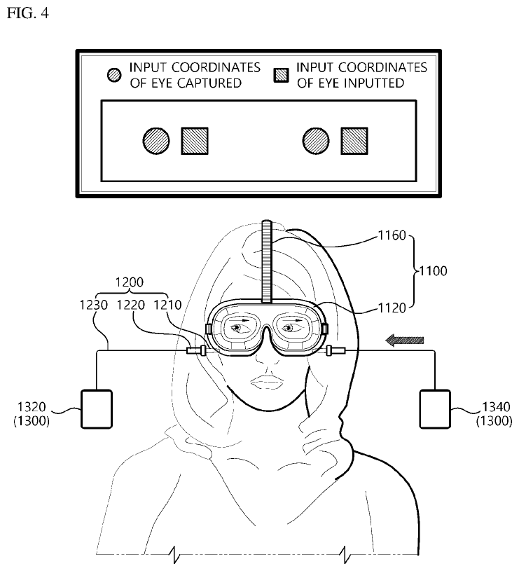

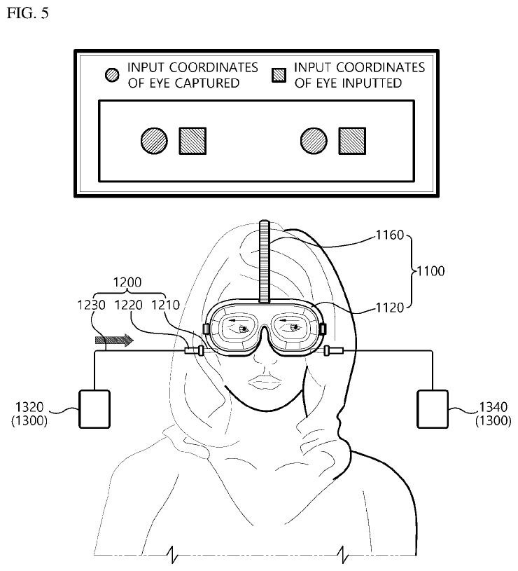

Referring to FIGS. 4 and 5, when the transmitted center coordinates of the eye are deviated from the center coordinates of the eye inputted in advance, the control part 1500 operates the heat source supply part 130 to increase the temperature of the first temperature-regulating pipe 1200a inserted into one ear of the examiner or decrease the temperature of the second temperature-regulating pipe 1200b inserted into the other ear of the examiner in order to move the eye of the captured image to the center coordinates of the eye inputted in advance.

A process in which the control part 1500 regulates the temperature of the first temperature-regulating pipe 1200a and the second temperature-regulating pipe 1200b according to the center coordinates of the eye transmitted to the control part 1500 and the center coordinates of the eye inputted to the control part 1500 in advance is as follows.

Referring to FIG. 4, when the center coordinates of the eye captured by the eye movement capturing means 1140 are located to the left side of the center coordinates of the eye inputted in advance, the control part 1500 operates the heat source supply part 1300 to decrease the temperature of the second temperature-regulating pipe 1200b. The control part 1500 operates the decreasing heat source supply part 1340 of the heat source supply part 1300 connected to the second temperature-regulating pipe 1200b to decrease the temperature of the second temperature-regulating pipe 1200b.

Referring to FIG. 5, when the center coordinates of the eye captured by the eye movement capturing means 1140 are located to the right side of the center coordinates of the eye inputted in advance, the control part 1500 operates the heat source supply part 1300 to increase the temperature of the first temperature-regulating pipe 1200a. The control part 1500 operates the increasing heat source supply part 1320 of the heat source supply part 1300 connected to the first temperature-regulating pipe 1200a to decrease the temperature of the first temperature-regulating pipe 1200a.

Preferably, when the temperature of the second temperature-regulating pipe 1200a is decreased, the control part 1500 decreases the temperature of the second temperature-regulating pipe 1200b to 20° C. Preferably, when the temperature of the first temperature-regulating pipe 1200a is increased, the control part 1500 increase the temperature of the first temperature-regulating pipe 1200a to 44° C. When the temperature of the second temperature-regulating pipe 1200b is less than 20° C. or the temperature of the first temperature-regulating pipe 1200a is 44° C. or more, the eardrum or the like is damaged. Accordingly, this causes a problem in that the function of the ear deteriorates.

Therefore, the control part controls the heat source supply part to regulate the temperature of the temperature-regulating pipe, so that quantitative caloric testing can be performed. Since the temperature-regulating pipe with high thermal conductivity is used instead of water, it is possible to minimize the stimulation to the ear of the examinee. The temperature-regulating pipe inserted into the ear of the examinee can be easily fixed using the fixing main body. Since eye movement is analyzed using the eye movement capturing means and the control part and the temperature inside the ear is controlled using the heat source supply part and the temperature-regulating pipe, repeated rehabilitation treatment is possible by artificially controlling the eye movement of the examinee or patient with ocular deviation. In addition, the symptoms of stroke patients are improved.

The present invention has been described with reference to the embodiments illustrated in the drawings, but this is only an example. It will be understood by those of ordinary skill in the art that various modifications and equivalents thereto may be made thereto. Therefore, the true technical protection scope of the present invention should be defined by the technical spirit of the appended claims.

### INDUSTRIAL APPLICABILITY

The present invention is usable in a device for diagnosing and treating ocular deviation.

1. A device for diagnosing and treating ocular deviation, comprising:
an eye mask which is worn by an examinee, and in which an eye movement capturing means for capturing the eye movement of the examinee is provided;
a temperature-regulating pipe which is inserted into the ear of the examinee and which increases or decreases the temperature inside the ear of the examinee;
a heat source supply part for supplying a heat source to the temperature-regulating pipe from the outside in order to increase and decrease the temperature of the temperature-regulating pipe;
a fixing main body which is worn by the examinee, and which has a mounting hole, in which the temperature-regulating pipe is mounted, in order to fix the temperature-regulating pipe inserted into the ear of the examinee; and
a control part, which is connected to the eye movement capturing means and the heat source supply part, receives an image of the eye captured by the eye movement capturing means, and extracts center coordinates from the transmitted image of the eye to compare same with the center coordinates of the eye inputted in advance, and then operates the heat source supply part to control the temperature of the temperature-regulating pipe in order to move the eye to the center coordinates of the eye inputted in advance.

2. The device of claim 1, wherein the eye mask comprises:
a mask main body which is worn around eyes of the examinee and which provides a dark environment for capturing eye movement;
an eye movement capturing means which is provided inside the mask main body and which captures the eye movement; and
a fixing means which is connected to the mask main body and which fixes the mask main body to a head of the examinee,
wherein the eye movement capturing means uses an infrared camera capable of capturing the eye movement even in the dark environment.

3. The device of claim 1, wherein the temperature-regulating pipe comprises:
a first temperature-regulating pipe inserted into one ear of the examinee; and
a second temperature-regulating pipe inserted into the other ear of the examinee,
wherein the first temperature-regulating pipe and the second temperature-regulating pipe comprise:
an insertion main body which is inserted into the ear of the examinee and which regulates the temperature in the ear of the examinee by transmitting the temperature of the heat source supplied from the heat source supply part;
a main body case which surrounds the insertion main body from the outside; and
a connecting member which connects the insertion main body and the heat source supply part.

4. The device of claim 3, wherein the insertion main body and the connecting member have a double pipe structure.

5. The device of claim 4, wherein the insertion main body comprises:
a main body inlet pipe into which the heat source supplied from the heat source supply part and the connecting member is introduced; and
a main body circulation pipe which circulates the heat source introduced into the main body inlet pipe,
wherein the connecting member comprises:
a connection inlet pipe which is connected to the main body inlet pipe and which supplies the heat source supplied from the heat source supply part to the main body inlet pipe; and
a connection circulation pipe which is connected to the main body circulation pipe and which circulates the heat source moving through the main body circulation pipe to the heat source supply part,
wherein the main body inlet pipe and the connection inlet pipe are located inside the main body circulation pipe and the connection circulation pipe.

6. The device of claim 5, wherein the insertion main body is made of a polymer material having a high heat transfer rate and a coefficient of thermal expansion similar to that of silicone.

7. The device of claim 1, wherein the fixing main body comprises:
a front head plate which is disposed in front of a head of the examinee;
a back head plate which is disposed behind the head of the examinee to correspond to the front head plate;
a first side connecting plate and a second side connecting plate which are disposed on both sides of the head of the examinee and which connect the front head plate and the rear head plate arranged to be spaced apart from each other;
a wearing belt which connects the first side connecting plate and the second side connecting plate; and
a fixing frame which is mounted on the first side connecting plate and the second side connecting plate and which fixes the temperature-regulating pipe in a state of being inserted into the ear of the examinee,
wherein the fixing frame is vertically moved from the first side connecting plate and the second side connecting plate.

8. The device of claim 7, wherein the fixing frame comprises:
a mounting body having one end mounted on the first side connecting plate and the second side connecting plate;
an elevating rod which is provided to be spaced apart from the other end of the mounting body;
an elevating main body which elevates in the elevating rod and has a through hole through which the elevating rod passes;
an elevating regulating member which is mounted on both sides of the elevating body and which regulates the elevation of the elevating main body; and
a mounting main body which is rotatably mounted to a mounting hole formed in the central portion of the elevating main body and on which the temperature-regulating pipe is mounted.

9. The device of claim 8, wherein a through hole is formed in the central portion of the mounting main body,
wherein the mounting main body comprises:
a mounting end which has a spherical circumferential surface and is rotatably mounted to the mounting hole;
a seating end which is connected to one side of the mounting end and on which the temperature-regulating pipe inserted into the through hole is seated; and
a separation preventing end which is provided at an end of the seating end and which prevents separation of the temperature-regulating pipe seated on the seating end,
wherein the separation preventing end is rotatably coupled to the seating end,
wherein the seating end is provided with a rotation regulating member for regulating the rotation of the separation preventing end.
